SEQUENCE LISTING (73 sequences)

SEQ ID NO: 1 - protein, 388 aa, Homo sapiens
MKTLIAAYSGVLRGERQAEADRSQRSHGGPALSREGSGRWGTGSSILSALQDLFSVTWLNRSKVEKQLQVISVLQWVLSFLVLGVACSAILMYIFCTDCWLIAVLYFTWLVFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVSRDTIDYLLSKNGSGNAIIIVVGGAAESLSSMPGKNAVTLRNRKGFVKLALRHGADLVPIYSFGENEVYKQVIFEEGSWGRWVQKKFQKYIGFAPCIFHGRGLFSSDTWGLVPYSKPITTVVGEPITIPKLEHPTQQDIDLYHTMYMEALVKLFDKHKTKFGLPETEVLEVN

SEQ ID NO: 2 - protein, 329 aa, Homo sapiens
MAHSKQPSHFQSLMLLQWPLSYLAIFWILQPLFVYLLFTSLWPLPVLYFAWLFLDWKTPERGGRRSAWVRNWCVWTHIRDYFPITILKTKDLSPEHNYLMGVHPHGLLTFGAFCNFCTEATGFSKTFPGITPHLATLSWFFKIPFVREYLMAKGVCSVSQPAINYLLSHGTGNLVGIVVGGVGEALQSVPNTTTLILQKRKGFVRTALQHGAHLVPTFTFGETEVYDQVLFHKDSRMYKFQSCFRRIFGFYCCVFYGQSFCQGSTGLLPYSRPIVTVVGEPLPLPQIEKPSQEMVDKYHALYMDALHKLFDQHKTHYGCSETQKLFFLZ

SEQ ID NO: 3 - protein, 338 aa, Homo sapiens
MAFFSRLNLQEGLQTFFVLQWIPVYIFLGAIPILLIPYFLLFSKFWPLAVLSLAWLTYDWNTHSQGGRRSAWVRNWTLWKYFRNYFPVKLVKTHDLSPKHNYIIANHPHGILSFGVFINFATEATGIARIFPSITPFVGTLERIFWIPIVREYVMSMGVCPVSSSALKYLLTQKGSGNAVVIVVGGAAEALLCRPGASTLFLKQRKGFVKMALQTGAYLVPSYSFGENEVFNQETFPEGTWLRLFQKTFQDTFKKILGLNFCTFHGRGFTRGSWGFLPFNRPITTVVGEPLPIPRIKRPNQKTVDKYHALYISALRKLFDQHKVEYGLPETQELTITZ

SEQ ID NO: 4 - protein, 334 aa, Homo sapiens
MLLPSKKDLKTALDVFAVFQWSFSALLITTTVIAVNLYLVVFTPYWPVTVLILTWLAFDWKTPQRGGRRFTCVRHWRLWKHYSDYFPLKLLKTHDICPSRNYILVCHPHGLFAHGWFGHFATEASGFSKIFPGITPYILTLGAFFWMPFLREYVMSTGACSVSRSSIDFLLTHKGTGNMVIVVIGGLAECRYSLPGSSTLVLKNRSGFVRMALQHGVPLIPAYAFGETDLYDQHIFTPGGFVNRFQKWFQSMVHIYPCAFYGRGFTKNSWGLLPYSRPVTTIVGEPLPMPKIENPSQEIVAKYHTLYIDALRKLFDQHKTKFGISETQELEIIZ

SEQ ID NO: 5 - protein, 335 aa, Homo sapiens
MKVEFAPLNIQLARRLQTVAVLQWVLSFLTGPMSIGITVMLIIHNYLFLYIPYLMWLYFDWHTPERGGRRSSWIKNWTLWKHFKDYFPIHLIKTQDLDPSHNYIFGFHPHGIMAVGAFGNFSVNYSDFKDLFPGFTSYLHVLPLWFWCPVFREYVMSVGLVSVSKKSVSYMVSKEGGGNISVIVLGGAKESLDAHPGKFTLFIRQRKGFVKIALTHGASLVPVVSFGENELFKQTDNPEGSWIRTVQNKLQKIMGFALPLFHARGVFQYNFGLMTYRKAIHTVVGRPIPVRQTLNPTQEQIEELHQTYMEELRKLFEEHKGKYGIPEHETLVLKZ

SEQ ID NO: 6 - protein, 335 aa, Homo sapiens
MVEFAPLFMPWERRLQTLAVLQFVFSFLALAEICTVGFIALLFTRFWLLTVLYAAWWYLDRDKPRQGGRHIQAIRCWTIWKYMKDYFPISLVKTAELDPSRNYIAGFHPHGVLAVGAFANLCTESTGFSSIFPGIRPHLMMLTLWFRAPFFRDYIMSAGLVTSEKESAAHILNRKGGGNLLGIIVGGAQEALDARPGSFTLLLRNRKGFVRLALTHGAPLVPIFSFGENDLFDQIPNSSGSWLRYIQNRLQKIMGISLPLFHGRGVFQYSFGLIPYRRPITTVVGKPIEVQKTLHPSEEEVNQLHQRYIKELCNLFEAHKLKFNIPADQHLEFCZ

SEQ ID NO: 7 - protein, 342 aa, Homo sapiens
MGVATTLQPPTTSKTLQKQHLEAVGAYQYVLTFLFMGPFFSLLVFVLLFTSLWPFSVFYLVWLYVDWDTPNQGGRRSEWIRNRAIWRQLRDYYPVKLVKTAELPPDRNYVLGAHPHGIMCTGFLCNFSTESNGFSQLFPGLRPWLAVLAGLFYLPVYRDYIMSFGLCPVSRQSLDFILSQPQLGQAVVIMVGGAHEALYSVPGEHCLTLQKRKGFVRLALRHGASLVPVYSFGENDIFRLKAFATGSWQHWCQLTFKKLMGFSPCIFWGRGLFSATSWGLLPFAVPITTVVGRPIPVPQRLHPTEEEVNHYHALYMTALEQLFEEHKESCGVPASTCLTFIZ

SEQ ID NO: 8 - DNA, 28 bp, artificial sequence
  5′ forward primer
gaattcatggctcattccaagcagccta

SEQ ID NO: 9 - DNA, 22 bp, artificial sequence
  5′ forward primer
atggctcattccaagcagccta

SEQ ID NO: 10 - DNA, 26 bp, artificial sequence
  5′ forward primer
ttgcctccctcaggctcccgagaatc

SEQ ID NO: 11 - DNA, 28 bp, artificial sequence
  5′ forward primer
gaattcatggctcattccaagcagccta

SEQ ID NO: 12 - DNA, 22 bp, artificial sequence
  5′ forward primer
atggctcattccaagcagccta

SEQ ID NO: 13 - DNA, 28 bp, artificial sequence
  5′ forward primer
gaattcatggctcattccaagcagccta

SEQ ID NO: 14 - DNA, 22 bp, artificial sequence
  5′ forward primer
atggctcattccaagcagccta

SEQ ID NO: 15 - DNA, 20 bp, artificial sequence
  3′ reverse primer
gtctctgagcagccatagtg

SEQ ID NO: 16 - DNA, 21 bp, artificial sequence
  3′ reverse primer
cgtaatggggaaatagtccct

SEQ ID NO: 17 - DNA, 22 bp, artificial sequence
  3′ reverse primer
cctgggcatgcagtaccttcat

SEQ ID NO: 18 - DNA, 31 bp, artificial sequence
  3′ reverse primer
gcggccgctcacaggaaaaacagcttttggg

SEQ ID NO: 19 - DNA, 20 bp, artificial sequence
  3′ reverse primer
gtctctgagcagccatagtg

SEQ ID NO: 20 - DNA, 20 bp, artificial sequence
  3′ reverse primer
gtctctgagcagccatagtg

SEQ ID NO: 21 - DNA, 21 bp, artificial sequence
  3′ reverse primer
cgtaatggggaaatagtccct

SEQ ID NO: 22 - DNA, 24 bp, artificial sequence
  5′ forward primer
atggctttcttctcccgactgaat

SEQ ID NO: 23 - DNA, 20 bp, artificial sequence
  5′ forward primer
ctggtgaagactcatgatct

SEQ ID NO: 24 - DNA, 24 bp, artificial sequence
  5′ forward primer
atggctttcttctcccgactgaat

SEQ ID NO: 25 - DNA, 24 bp, artificial sequence
  5′ forward primer
atggctttcttctcccgactgaat

SEQ ID NO: 26 - DNA, 20 bp, artificial sequence
  5′ forward primer
ctggtgaagactcatgatct

SEQ ID NO: 27 - DNA, 20 bp, artificial sequence
  3′ reverse primer
cactgatgtagagtgcgtga

SEQ ID NO: 28 - DNA, 20 bp, artificial sequence
  3′ reverse primer
tcaaggctgagctactcaca

SEQ ID NO: 29 - DNA, 20 bp, artificial sequence
  3′ reverse primer
tcaaggctgagctactcaca

SEQ ID NO: 30 - DNA, 20 bp, artificial sequence
  3′ reverse primer
cactgatgtagagtgcgtga

SEQ ID NO: 31 - DNA, 20 bp, artificial sequence
  3′ reverse primer
tcaaggctgagctactcaca

SEQ ID NO: 32 - DNA, 29 bp, artificial sequence
  5′ forward primer
gaattcatgatcttgccctctaagaagga

SEQ ID NO: 33 - DNA, 20 bp, artificial sequence
  5′ forward primer
tctgaagactcatgacatct

SEQ ID NO: 34 - DNA, 22 bp, artificial sequence
  5′ forward primer
agccactgggacgctgggcaca

SEQ ID NO: 35 - DNA, 29 bp, artificial sequence
  5′ forward primer
gaattcatgctcttgccctctaagaagga

SEQ ID NO: 36 - DNA, 20 bp, artificial sequence
  5′ forward primer
tctgaagactcatgacatct

SEQ ID NO: 37 - DNA, 29 bp, artificial sequence
  5′ forward primer
gaattcatgctcttgccctctaagaagga

SEQ ID NO: 38 - DNA, 20 bp, artificial sequence
  5′ forward primer
tctgaagactcatgacatct

SEQ ID NO: 39 - DNA, 30 bp, artificial sequence
  3′ reverse primer
gcggccgctcaaattatctccagctcctgg

SEQ ID NO: 40 - DNA, 20 bp, artificial sequence
  3′ reverse primer
atgtgctgatcatagaggtc

SEQ ID NO: 41 - DNA, 22 bp, artificial sequence
  3′ reverse primer
tgaaggctactggggatgtctg

SEQ ID NO: 42 - DNA, 30 bp, artificial sequence; identical to SEQ ID NO: 39
  3′ reverse primer

SEQ ID NO: 43 - DNA, 20 bp, artificial sequence
  3′ reverse primer
atgtgctgatcatagaggtc

SEQ ID NO: 44 - DNA, 30 bp, artificial sequence; identical to SEQ ID NO: 39
  3′ reverse primer

SEQ ID NO: 45 - DNA, 20 bp, artificial sequence
  3′ reverse primer
atgtgctgatcatagaggtc

SEQ ID NO: 46 - DNA, 20 bp, artificial sequence
  5′ forward primer
atgaaggtagagtttgcacc

SEQ ID NO: 47 - DNA, 20 bp, artificial sequence
  5′ forward primer
ctgtgatgctgatcatacac

SEQ ID NO: 48 - DNA, 20 bp, artificial sequence
  5′ forward primer
atgaaggtagagtttgcacc

SEQ ID NO: 49 - DNA, 20 bp, artificial sequence
  5′ forward primer
ctgtgatgctgatcatacac

SEQ ID NO: 50 - DNA, 20 bp, artificial sequence
  3′ reverse primer
tcattttaaaacaagagtct

SEQ ID NO: 51 - DNA, 20 bp, artificial sequence
  3′ reverse primer
agagtgaactttccaggatg

SEQ ID NO: 52 - DNA, 20 bp, artificial sequence
  3′ reverse primer
tcattttaaaacaagagtct

SEQ ID NO: 53 - DNA, 20 bp, artificial sequence
  3′ reverse primer
agagtgaactttccaggatg

SEQ ID NO: 54 - DNA, 28 bp, artificial sequence
  5′ forward primer
gaattcatggtagagttcgcgcccttgt

SEQ ID NO: 55 - DNA, 20 bp, artificial sequence
  5′ forward primer
tgcagacacttgctgtccta

SEQ ID NO: 56 - DNA, 20 bp, artificial sequence
  5′ forward primer
tgcagacacttgctgtccta

SEQ ID NO: 57 - DNA, 28 bp, artificial sequence
  5′ forward primer
gaattcatggtagagttcgcccccttgt

SEQ ID NO: 58 - DNA, 20 bp, artificial sequence
  5′ forward primer
tgcagacacttgctgtccta

SEQ ID NO: 59 - DNA, 29 bp, artificial sequence
  3′ reverse primer
gcggccgctcagcagaactccaagtgctg

SEQ ID NO: 60 - DNA, 20 bp, artificial sequence
  3′ reverse primer
atgcagcgtcttctgtacct

SEQ ID NO: 61 - DNA, 21 bp, artificial sequence
  3′ reverse primer
cagagattacatcatgtctgc

SEQ ID NO: 62 - DNA, 29 bp, artificial sequence
  3′ reverse primer
gcggccgctcagcagaactccaagtgctg

SEQ ID NO: 63 - DNA, 20 bp, artificial sequence
  3′ reverse primer
atgcagcgtcttctgtacct

SEQ ID NO: 64 - DNA, 20 bp, artificial sequence
  5′ forward primer
cttgcagaagcagcatctag

SEQ ID NO: 65 - DNA, 24 bp, artificial sequence
  5′ forward primer
atgggagttgccacaaccctgcag

SEQ ID NO: 66 - DNA, 20 bp, artificial sequence
  5′ forward primer
cttgcagaagcagcatctag

SEQ ID NO: 67 - DNA, 24 bp, artificial sequence
  5′ forward primer
atgggagttgccacaaccctgcag

SEQ ID NO: 68 - DNA, 20 bp, artificial sequence
  5′ forward primer
cttgcagaagcagcatctag

SEQ ID NO: 69 - DNA, 19 bp, artificial sequence
  3′ reverse primer
gcgtgatagtgattgactt

SEQ ID NO: 70 - DNA, 19 bp, artificial sequence
  3′ reverse primer
gcgtgatagtgattgactt

SEQ ID NO: 71 - DNA, 20 bp, artificial sequence
  3′ reverse primer
catgatgtagtcgcgataga

SEQ ID NO: 72 - DNA, 19 bp, artificial sequence
  3′ reverse primer
gcgtgatagtgattgactt

SEQ ID NO: 73 - DNA, 20 bp, artificial sequence
  3′ reverse primer
catgatgtagtcgcgataga

(19) United States
(12) Patent Application Publication
(10) Pub. No.: US 2006/0100146 A1
(43) Pub. Date: May 11, 2006

(54) AWAT-related methods and articles

(76) Inventors: Stephen L. Sturley, New York, NY (US); Aaron Turkish, New York, NY (US); Jeffrey T. Billheimer, West Chester, PA (US); Debra Cromley, Pittsgrove, NJ (US)

Correspondence Address: COOPER & DUNHAM, LLP, 1185 AVENUE OF THE AMERICAS, NEW YORK, NY, 10036, US

(21) Appl. No.: 11/204,427

(22) Filed: Aug. 15, 2005

Related U.S. Application Data
(60) Provisional application No. 60/602,076, filed on Aug. 16, 2004.

(51) Int. Cl. A61K 38/26; A61K 31/685; A61K 31/553; A61K 31/366; A61K 31/19

(52) U.S. Cl. 514/12; 514/78; 514/457; 514/460; 514/557; 514/571; 514/356; 514/211.13

(57) ABSTRACT

This invention provides methods for treating a subject afflicted with acne or another sebaceous gland disorder comprising administering to the subject a therapeutically effective amount of an agent which inhibits acyl-CoA wax alcohol acyltransferase 1 (AWAT1) and/or acyl-CoA wax alcohol acyltransferase 2 (AWAT2), thereby treating the subject. This invention further provides related articles of manufacture and methods.

This application claims the benefit of U.S. Provisional Application No. 60/592,056 filed Aug. 16, 2004, the contents of which are hereby incorporated by reference.

This invention was made with support under Grant Numbers DK54320 and HL07343 from the National Institutes of Health. Accordingly, the United States Government has certain rights in this invention.

Throughout this application, various publications are referenced by numbers. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

In eukaryotes, the cytoplasmic storage of fatty acids in the form of triacylglycerol (TAG) serves to provide reservoirs for membrane formation and maintenance, lipoprotein trafficking, detoxification of fatty acid and alcohol substrates, epidermal integrity and fuel in times of stress or nutrient deprivation (reviewed (1-3)). By contrast, the subcellular and extracellular accumulation of TAG and other neutral lipids has been linked to several human disease states such as diabetes mellitus (4), obesity (4), atherosclerosis (5,6) and non-alcoholic fatty liver disease (7). Understanding the metabolic pathways of triglyceride synthesis and the roles of the enzymes involved in these reactions may hasten the development of therapeutic interventions by clarifying the pathophysiological processes of these diseases. Diacylglycerol is esterified to triglyceride by an acyl-CoA:diacylglycerol acyltransferase (DGAT) reaction. There are at least two independent mammalian enzymes known to catalyze this reaction, DGAT1 (8) and DGAT2 (9,10).

DGAT1 is a member of the acyl-CoA:cholesterol acyltransferase (ACAT) gene family with high levels of expression in human small intestine, colon, testis and skeletal muscle. Mice lacking DGAT1 are surprisingly healthy with normal serum TAG but are resistant to diet-induced obesity, and have impaired sebaceous gland secretion (11-14).

Subsequent to the discovery of DGAT1, DGAT2, the original member of a second human DGAT family, was identified by sequence similarity to lipid droplet proteins purified from Mortierella ramanniana, an oleaginous fungus (10). When expressed in insect cells, DGAT2 produces robust DGAT activity. DGAT2 shares no sequence similarity with DGAT1 and exhibits widespread expression in humans, with particularly high levels in liver and adipose tissue. Recently it has been shown that DGAT2^{-/-} mice are severely depleted of triglycerides in their tissues and plasma, and possess poor skin barrier function, leading to early death (15). DGAT1 was unable to fully compensate for the loss of DGAT2, suggesting different roles for the two enzymes, and that DGAT2 is the enzyme responsible for the majority of TAG synthesis in mice.

Similarly, DGA1, the sole member of the DGAT2 gene family in Saccharomyces cerevisiae, is responsible for a large portion of triglyceride synthesis in that model organism (16-18). This activity is complemented by that of LRO1 (an ortholog of mammalian lecithin cholesterol acyltransferase) which also esterifies diacylglycerol but uses phospholipids as the acyl donor (19,20). While their relative contribution varies with culture conditions, together they are responsible for about 98% of triglyceride synthesis in yeast. Deletion of ARE2, a steryl ester synthase, in conjunction with DGA1 and LRO1 eliminates detectable TAG synthesis in yeast (17).

### SUMMARY OF THE INVENTION

This invention provides a method for treating a subject afflicted with a disorder selected from the group consisting of a sebaceous gland disorder, obesity and atherosclerosis, comprising administering to the subject a therapeutically effective amount of an agent which inhibits acyl-CoA wax alcohol acyltransferase 1 (AWAT1) and/or acyl-CoA wax alcohol acyltransferase 2 (AWAT2), thereby treating the subject.

This invention further provides a method for treating a subject afflicted with acne comprising topically administering to an afflicted area on the subject's skin a therapeutically effective amount of an agent which inhibits AWAT1 and/or AWAT2, thereby treating the subject.

This invention further provides a method for inhibiting the activity of AWAT1 and/or AWAT2 in a sebaceous gland comprising contacting the gland with a therapeutically effective amount of an agent which inhibits AWAT1 and/or AWAT2, thereby inhibiting the activity of AWAT1 and/or AWAT2 in the sebaceous gland.

This invention further provides an article of manufacture comprising (i) a packaging material having therein an agent which inhibits AWAT1 and/or AWAT2 admixed with a pharmaceutical carrier, and (ii) instructions for using the agent to treat a subject afflicted with a disorder selected from the group consisting of a sebaceous gland disorder, obesity and atherosclerosis.

This invention further provides an article of manufacture comprising (i) a packaging material having therein an agent which inhibits AWAT1 and/or AWAT2 admixed with a pharmaceutical carrier for topical administration, and (ii) instructions for using the agent to treat a subject afflicted with acne.

This invention further provides a method for determining whether an agent inhibits AWAT1 activity comprising (a) contacting the agent with AWAT1 under conditions permitting AWAT1 activity in the absence of the agent; (b) measuring the activity of AWAT1 in the presence of the agent; and (c) determining whether the activity measured in step (b) is less than the activity in the absence of the agent, whereby AWAT1 activity which is less in the presence of the agent than in its absence indicates that the agent is an AWAT1 inhibitor.

This invention further provides a method for determining whether an agent inhibits AWAT2 activity comprising (a) contacting the agent with AWAT2 under conditions permitting AWAT2 activity in the absence of the agent; (b) measuring the activity of AWAT2 in the presence of the agent; and (c) determining whether the activity measured in step (b) is less than the activity in the absence of the agent, whereby AWAT2 activity which is less in the presence of the agent than in its absence indicates that the agent is an AWAT2 inhibitor.

This invention further provides a method for determining whether an agent known to inhibit AWAT1 specifically inhibits AWAT1 comprising (a) measuring the amount of AWAT1 inhibition in the presence of the agent under predetermined conditions; and (b) determining whether the amount of inhibition measured in step (a) is greater than the amount of inhibition by the agent, under the predetermined conditions, of AWAT2 and any other member of the DGAT2 family, whereby if the inhibition of AWAT1 is determined in step (b) to be greater, the agent specifically inhibits AWAT1.

Finally, this invention provides a method for determining whether an agent known to inhibit AWAT2 specifically inhibits AWAT2 comprising (a) measuring the amount of AWAT2 inhibition in the presence of the agent under predetermined conditions; and (b) determining whether the amount of inhibition measured in step (a) is greater than the amount of inhibition by the agent, under the predetermined conditions, of AWAT1 and any other member of the DGAT2 family, whereby if the inhibition of AWAT2 is determined in step (b) to be greater, the agent specifically inhibits AWAT2.

### BRIEF DESCRIPTION OF THE FIGURES

FIG. 1A Sequence alignments of the human DGAT2 family

Amino acid sequences of the DGAT2 family were aligned using the CLUSTALW multiple sequence alignment program. Dark and light shading indicate identity and similarity of residues, respectively. Conservation of putative active site residues based on mutagenesis of GPATs and LPAATs are indicated by an asterisk (*). Putative transmembrane domains are indicated by underlines.

FIG. 1B Dendrogram depicting the evolutionary relationship of the human DGAT2 gene family

A phylogenetic tree indicating the relatedness of DGAT2 family sequences was constructed using the CLUSTALW program. DGA2, hDC3 and hDC4 appear to be derived from hDGAT2 and are more closely related to each other than to the MGAT sub-family.

FIG. 1C Genomic structure spanning DGA2, hDC3 and hDC4 on the human X-chromosome

The representation is based on physical data from the NCBI, Ensembl, UCSC and Celera genome datasets. Shaded boxes depict exons. The locus of 3 genes spans about 200 Kbp with a significant density of predicted genes in the intergenic regions. Extended intergenic regions are indicated by the line-breaks. ATG represents the likely translational starts, with the direction of transcription indicated by the arrows.

FIGS. 2A-2C: Tissue expression of the human DGAT2 gene family

(A) Analysis of the MGAT sub-family tissue expression was performed via standard (MGAT3) or nested PCR (MGAT 1 & 2) using human cDNA obtained as part of a Quick Screen cDNA panel of human tissues obtained from CLONTECH and primers specific for each gene (Table 4). Splice variants for MGAT1 were gel isolated, purified and sequenced. (B) Analysis of human DGA2, hDC3 and hDC4 tissue expression was performed via nested PCR using the same cDNA tissue panel and method as described above. A splice variant of hDC4 was also isolated, purified and sequenced. (C) Nested RT-PCR was performed on mRNA using primers specific for each gene (Table 4) to determine expression of the DGAT2 gene family in adipose tissue. Note similar splice variation found in hDC4 and MGAT1 as described above in addition to splice variant visualized in MGAT2.

FIG. 3: Northern blot analysis of human DGA2, hDC3 and hDC4 expression in TG null strains of yeast

10 μg of RNA from cultures of transformed TG null yeast strains grown in either dextrose (D) or galactose (G) was resolved on a 1.2% agarose, formaldehyde gel, transferred to nylon membrane, and hybridized in Quik-Hyb buffer at 65° C. for 1 hour with a random-hexamer primed, [³²P]dCTP-labeled gene specific probe. After washing in 0.1×SSC+0.1% SDS at 65° C. for 30 minutes, the membrane was exposed to X-Ray film. Molecular weight markers were supplied by CLONTECH. The predicted length of DGA2 is 987 bp, hDC3 is 1014 bp, and hDC4 is 1002 b.p.

FIGS. 4A-4B: Human DGAT2, DGA2, hDC3 and hDC4 synthesize triglyceride in yeast in vivo

TG null yeast strains transformed with human DGAT2, DGA2, hDC3, hDC4, and a vector harboring no insert (VC) were grown to log phase and pulse labeled with [³H] palmitate (A) and [³H] oleate (B), as described under Experimental Procedures. Asterisks denote statistically significant (p<0.05) differences compared to VC.

FIGS. 5A-5B: Expression of DGA2, hDC3 and hDC4 in human skin

(A) Analysis of tissue expression of the human DGAT2 gene family in skin was performed via standard PCR using skin cDNA and primers specific for each gene (see Table 4) as described in Experimental Procedures. (B) In situ hybridization was performed on freshly cut human scalp frozen sections using anti-sense probes for DGA2, hDC3 and hDC4 and counterpart sense probe controls as described in Experimental Procedures. Gene specific PCR primer pairs (Table 4) were used to amplify templates from the pRS423-GP vector harboring each cDNA insert.

### DETAILED DESCRIPTION OF THE INVENTION

Definitions

As used in this application, except as otherwise expressly provided herein, each of the following terms shall have the meaning set forth below.

As used herein, “acne” shall mean any type of acne, including, without limitation, common acne, acne albidia, acne artificialis, acne bacillus, acne cacheticorum, acne cilaris, acne conglobata, acne cosmetica, acne decalvans, acne erythematosa, acne frontalis, acne fulminans, acne generalis, acne hypertrophica, acne indurate, acne keloid, acne keratosa, acne medicamentosa, acne necrotica, acne neonatorum, acne papulosa, acne punctata, acne pustulosa, acne rosacea, acne scrofulosorum, acne simplex, acne syphilitica, acne urticata, acne varioliformis, acne venenata, and acne vulgaris.

As used herein, “administering” shall mean delivering in a manner which is effected or performed using any of the various methods and delivery systems known to those skilled in the art. Administering can be performed, for example, intravenously, orally, via implant, transmucosally, transdermally, intramuscularly, or subcutaneously. Specifically envisioned is topical administration. “Administering” can also be performed, for example, once, a plurality of times, and/or over one or more extended periods.

As used herein, “agent” shall include, without limitation, an organic compound, a nucleic acid, a polypeptide, a lipid, and a carbohydrate. Agents include, for example, agents which are known with respect to structure and/or function, and those which are not known with respect to structure or function.

As used herein, “AWAT1” shall mean acyl-CoA wax alcohol acyltransferase 1 and its synonym diacyl-glycerol acyltransferase 2 (DGA2).

As used herein, “AWAT2” shall mean acyl-CoA wax alcohol acyltransferase 2 and its synonym human DGAT candidate gene 4 (hDC4).

As used herein, the members of the “diacylgylcerol acyltransferase 2 (DGAT2) family” are diacylglycerol acyltransferase 2 (DGAT2), acyl-CoA monoacylglycerol acyltransferase 1 (MGAT1), acyl-CoA monoacylglycerol acyltransferase 2 (MGAT2), acyl-CoA monoacylglycerol acyltransferase 3 (MGAT3), AWAT1, AWAT2 and human DGAT candidate gene 3 (hDC3).

As used herein, “inhibiting” shall mean either lessening the activity and/or expression of AWAT1 and/or AWAT2, or preventing the activity and/or expression of AWAT1 and/or AWAT2 entirely.

As used herein, “pharmaceutically acceptable carriers” include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

Dermal delivery systems for topical administration include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

As used herein, “sebaceous gland disorder” shall mean any disorder caused by, and/or whose symptoms comprise, dysfunction of the sebaceous gland.

As used herein, “selectively inhibit”, with respect to an enzyme, shall mean to inhibit that enzyme more than any other enzyme. Such inhibition can occur, for example, via inhibiting the enzyme's activity (e.g. via a competitive inhibitor) or via inhibiting expression of the enzyme in a cell. For example, an agent which inhibits the activity of AWAT1 more than it inhibits the activity of AWAT2 or any other enzyme selectively inhibits AWAT1. Likewise, for example, an agent which inhibits the expression of AWAT2 more than it inhibits the expression of AWAT1 or any other enzyme selectively inhibits AWAT2.

As used herein, “subject” shall mean any animal, such as a primate, mouse, rat, guinea pig or rabbit. In the preferred embodiment, the subject is a human.

As used herein, “therapeutically effective amount” means an amount sufficient to treat a subject afflicted with a disorder or a complication associated with a disorder.

As used herein, “treating” a subject afflicted with a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent.

### EMBODIMENTS OF THE INVENTION

This invention provides a method for treating a subject afflicted with a disorder selected from the group consisting of a sebaceous gland disorder, obesity and atherosclerosis, comprising administering to the subject a therapeutically effective amount of an agent which inhibits acyl-CoA wax alcohol acyltransferase 1 (AWAT1) and/or acyl-CoA wax alcohol acyltransferase 2 (AWAT2), thereby treating the subject.

In one embodiment of the instant method, the sebaceous gland disorder is selected from the group consisting of acne, rosacea, perioral dermatitis, sebaceous cysts, seborrhea and alopecia. In another embodiment of this method, the agent can selectively inhibit AWAT1. In yet another embodiment of this method, the agent can selectively inhibit AWAT2.

The agent which inhibits AWAT1 and/or AWAT2 can be selected, for example, from the group consisting of magnesium, 2-bromooctanoate, an amidepsine, gemfibrozil, tetradecylthioacetic acid, isochromophilones, niacin, a tanshinone, cochiloquinones A and A1, fenofibrate, simvastatin, an n-3 polyunsaturated fatty acid, a phospholipid with a polar head group modified with monomethylethanolamine and/or dimethlyethanolamine, epidermal growth factor, a quinolone alkaloid, abscisic acid, an alkylaryl polyether alcohol, 2-bromopalmitate, 2-bromopalmitoyl-CoA, a lysophospholipid, glucagon, adrenaline, a chalcone, a roselipin, a prenylflavonoid, a polyacetylene, a benzoxiperone, and N-(7,10-dimetyl-11-oxo-10,11-dihydro-dibenzo[b,f][1,4]oxazepin-2-yl)-4-hydroxy-benzamide.

In one embodiment of this method, the subject is a human.

This invention further provides a method for treating a subject afflicted with acne comprising topically administering to an afflicted area on the subject's skin a therapeutically effective amount of an agent which inhibits AWAT1 and/or AWAT2, thereby treating the subject.

This invention further provides a method for inhibiting the activity of AWAT1 and/or AWAT2 in a sebaceous gland comprising contacting the gland with a therapeutically effective amount of an agent which inhibits AWAT1 and/or AWAT2, thereby inhibiting the activity of AWAT1 and/or AWAT2 in the sebaceous gland.

In one embodiment of this method, the sebaceous gland is a human sebaceous gland.

This invention further provides an article of manufacture comprising (i) a packaging material having therein an agent which inhibits AWAT1 and/or AWAT2 admixed with a pharmaceutical carrier, and (ii) instructions for using the agent to treat a subject afflicted with a disorder selected from the group consisting of a sebaceous gland disorder, obesity and atherosclerosis.

In one embodiment of this article, (i) the pharmaceutical carrier is for topical administration and (ii) the instructions are for using the agent to treat a subject having a sebaceous gland disorder selected from the group consisting of acne, rosacea, perioral dermatitis, sebaceous cysts, seborrhea and alopecia. In another embodiment of this article, the subject is a human.

This invention further provides an article of manufacture comprising (i) a packaging material having therein an agent which inhibits AWAT1 and/or AWAT2 admixed with a pharmaceutical carrier for topical administration, and (ii) instructions for using the agent to treat a subject afflicted with acne.

This invention further provides a method for determining whether an agent inhibits AWAT1 activity comprising (a) contacting the agent with AWAT1 under conditions permitting AWAT1 activity in the absence of the agent; (b) measuring the activity of AWAT1 in the presence of the agent; and (c) determining whether the activity measured in step (b) is less than the activity in the absence of the agent, whereby AWAT1 activity which is less in the presence of the agent than in its absence indicates that the agent is an AWAT1 inhibitor.

In one embodiment of this method, the AWAT1 is human AWAT1. In another embodiment of this method, the agent is known to inhibit at least one other member of the diacylglycerol acyltransferase 2 (DGAT2) family.

This invention further provides a method for determining whether an agent inhibits AWAT2 activity comprising (a) contacting the agent with AWAT2 under conditions permitting AWAT2 activity in the absence of the agent; (b) measuring the activity of AWAT2 in the presence of the agent; and (c) determining whether the activity measured in step (b) is less than the activity in the absence of the agent, whereby AWAT2 activity which is less in the presence of the agent than in its absence indicates that the agent is an AWAT2 inhibitor. In one embodiment of this method, the AWAT2 is human AWAT2.

This invention further provides a method for determining whether an agent known to inhibit AWAT1 specifically inhibits AWAT1 comprising (a) measuring the amount of AWAT1 inhibition in the presence of the agent under predetermined conditions; and (b) determining whether the amount of inhibition measured in step (a) is greater than the amount of inhibition by the agent, under the predetermined conditions, of AWAT2 and any other member of the DGAT2 family, whereby if the inhibition of AWAT1 is determined in step (b) to be greater, the agent specifically inhibits AWAT1.

Finally, this invention further provides a method for determining whether an agent known to inhibit AWAT2 specifically inhibits AWAT2 comprising (a) measuring the amount of AWAT2 inhibition in the presence of the agent under predetermined conditions; and (b) determining whether the amount of inhibition measured in step (a) is greater than the amount of inhibition by the agent, under the predetermined conditions, of AWAT1 and any other member of the DGAT2 family, whereby if the inhibition of AWAT2 is determined in step (b) to be greater, the agent specifically inhibits AWAT2.

Specific embodiments of one aspect of the instant invention explicitly set forth above are also envisioned mutates mutandis, where applicable, to the remaining aspects of this invention.

This invention is illustrated in the Experimental Details section which follows. This section is set forth to aid in an understanding of the invention but is not intended to, and should not be construed to, limit in any way the invention as set forth in the claims which follow thereafter.

### EXPERIMENTAL DETAILS

Summary

The esterification of alcohols such as sterols, diacylglycerols and monoacylglycerols with fatty acids represents the formation of both storage and cytoprotective molecules. Conversely, the overproduction of these molecules is associated with several disease pathologies, including atherosclerosis and obesity. The human acyl-CoA:diacylglycerol acyltransferase (DGAT) 2 gene superfamily comprises 7 members, four of which have been previously implicated in the synthesis of di- or tri-acylglycerol. The remaining 3 members comprise an X-linked locus and have not been characterized. The expression of DGAT2 and the 3 X-linked genes in Saccharomyces cerevisiae strains are described as virtually devoid of neutral lipids. All 4 gene products mediate the synthesis of triacylglycerol; however, two of the X-linked genes act as acyl-CoA wax alcohol acyltransferases (AWAT 1 and 2) that predominantly esterify long chain (wax) alcohols with acyl-CoA derived fatty acids to produce wax esters. AWAT1 and AWAT2 have very distinct substrate preferences in terms of alcohol chain length and fatty acyl saturation. The enzymes are expressed in many human tissues but predominate in skin. In situ hybridizations demonstrate a differentiation specific expression pattern within the human sebaceous gland for the two AWAT genes, consistent with a significant role in the composition of sebum.

The human DGAT2 gene family consists of seven members: DGAT2 (10), 3 acyl-CoA monoacylglycerol acyltransferases (MGATs 1, 2, & 3 (21-23)), and the three genes characterized by this study. Based on expression patterns, MGAT2 and MGAT3 were proposed as the major mediators of the intestinal MAG pathway. This pathway predominates in the intestine, whereby 2-monoacylglycerol, a product of partial lipolysis of triglyceride, is re-esterified using a fatty acyl-CoA to produce diacylglycerol. Moreover, the substrate preference of MGAT3 (49% sequence identity with human DGAT2) for 2-monoacylglycerol, compared to MGAT1 and MGAT2, which primarily use the sn-1 and sn-3 stereoisomers of MAG, implicates it as the major MGAT involved in intestinal fat absorption. Interestingly, however, there is no mouse ortholog of MGAT3. Murine MGAT2 is 46% identical to DGAT2 and is predominantly expressed in small intestine; however, human MGAT2 is more widespread (22, 24-26). The MGATs also exhibit some DGAT activity and so the precise physiologic roles of these members of the DGAT2 gene family remain to be determined. Two other DGAT2 family members were originally reported (human DGAT candidate (hDC) genes three and four (10)) and herein the final member of the human family is described, which is temporarily name DGA2. cDNAs for these three genes were isolated and expressed and the substrate specificity, tissue expression patterns and genealogic relationships of the enzymes were defined, relative to human DGAT2. Markedly, all 3 enzymes are capable of esterifying DAG. However, the preferred alcohol substrates for two of them are long chain alcohols leading to the production of wax esters. Therefore, these enzymes are renamed acyl-CoA wax alcohol acyltransferase (AWAT) 1 and 2.

Experimental Procedures

General

Molecular biology and yeast procedures were performed according to conventional protocols (27,28). Complete (yeast extract, peptone, dextrose; YPD), synthetic complete (SC) and selective media (±galactose for cDNA expression in yeast) were prepared as described (27). DNA modifying reagents were purchased from New England Biolabs. Oligonucleotide primers were synthesized by Invitrogen. Human cDNA obtained as part of a Quick Screen cDNA panel of human tissues was obtained from CLONTECH (BD Biosciences). Skin cDNA was obtained from Invitrogen. IMAGE clones were obtained from Invitrogen. The Qiaquick Gel Extraction Kit (Qiagen) and the Wizard PCR Preps DNA Purification System (Promega) were used to purify PCR products. The Prime-It random priming probe synthesis kit was obtained from Stratagene. [³²P]dCTP, [9,10-³H(N)] & [1-¹⁴C]oleic acid, and [9,10-³H(N)]palmitic acid were from Perkin Elmer Life Sciences. Automated DNA sequencing was performed at the Columbia University Cancer Center sequencing facility.

Identification, Isolation, and Construction of Expression Plasmids with Human DGA2, hDC3 and hDC4

Human expressed sequence tags, genomic sequences and predicted mRNA sequences for DGA2, hDC4 and hDC3 were obtained via a comprehensive BLAST search (29,30) through their homology to human DGAT2 (GenBank accession number AF384161). The resultant sequences were then used to predict the coding sequence of the candidate genes, analyzed, and compared using DNAStrider (31) and the CLUSTALW program in MacVector (32). Protein sequences were analyzed using the PROSITE and SMART analysis programs (33) (34). The full-length coding sequence for DGAT2 and hDC3 (GenBank accession number BG743707) were obtained as IMAGE cDNA clones in the pOTB7 (Clone ID 4644380) and pCMV-SPORT6 (Clone ID 4778300) vectors, respectively, and sequenced. Expression plasmids for the full length human DGAT2 and hDC3 were engineered by subcloning the cDNA fragments into the EcoRI and XmaI, and EcoRI sites, respectively, of the yeast galactose inducible vector, pRS423-GP, downstream of the GAL 1/10 promoter (35). Using predicted cDNA flanking regions, oligo primer pairs (Table 4) were designed to amplify the full-length coding region of DGA2 and hDC4 from thymus and lung cDNA, respectively. Amplification was performed via nested PCR using 2 ng of human cDNA, 10×PCR Buffer, 1.75 mM MgCl₂, 1.5 Units of Taq polymerase, (Taq DNA polymerase, Invitrogen), 1 μM oligo primers, and 0.4 Units of Pfu DNA Polymerase (Stratagene) in the Gene Amp PCR System 2400 (Applied Biosystems). PCR reactions were held at 94° C. for 5 min followed by 28 cycles of 94° C. for 15 s, 55° C. for 30 s and 72° C. for 1.5 min. PCR products were gel isolated, purified, TA cloned into the EcoRI and NotI sites of the pCR 2.1-TOPO vector (Invitrogen), and transformed into chemically competent Top10 E. coli (Rapid One Shot Chemical Transformation, Invitrogen). The full-length hDC4 was then sub cloned into the EcoRI site of the pRS423-GP vector downstream of the GAL 1/10 promoter. The full length DGA2 was sub cloned into the EcoRI and NotI sites of the pRS423-GP vector, also downstream to the GAL 1/10 promoter. The full-length cDNA of DGA2 and hDC4 were entirely sequenced.

Yeast Strains and Transformation

Strains of S. cerevisiae with triple deletions of the DGA1, LRO1 and ARE2 (SCY2056, dga1Δ::URA3 lro1Δ::URA3 are2::LEU2) genes have been previously described to possess almost 99% depletion of triglyceride synthesis capabilities (17). These strains were transformed with the human DGAT2, DGA2, hDC3 and hDC4 expression plasmids or pRS423-GP using lithium acetate followed by prototrophic selection (36).

Expression of DGA2, hDC3 and hDC4

RNA was prepared from transformed yeast strains grown in SC-His+2% galactose+1% raffinose or SC-His+2% dextrose (control). 10 μg of RNA from each culture was resolved on a 1.2% agarose, formaldehyde gel, transferred to nylon membrane (Hybond-N, Amersham), and hybridized in Quik-Hyb buffer (Stratagene) at 65° C. for 1 hour with a random-hexamer primed, [³²P]dCTP-labeled gene specific probe, generated via PCR (Table 1). Molecular weight markers were supplied by CLONTECH.

Lipid Synthesis

Metabolic pulse labeling in exponential phase was performed as previously described (17,19). A dilute inoculation (1/20-1/30) of a saturated culture grown in SC-His+2% dextrose, followed by washes and resuspension in SC-His+2% galactose+1% raffinose, was grown at 30° C. to early logarithmic phase (OD₆₀₀=0.3-0.4). 10 mL of cells were added to 0.01 μCi/mL of [³H] or [¹⁴C]oleate or 0.25 μCi/mL of [³H]palmitate and incubated at 30° C. for 1 hour with shaking, washed twice with 0.5% tergitol and once with dH₂O, and lyophilized. Lipids were extracted from dried cell pellets by cell wall hydrolysis (lyticase) and organic extraction (37) and were resolved by TLC in petroleum ether:diethyl ether:acetic acid (84:15:1) and the two-solvent resolution system as described (38). Each lane was cut according to lipid standards and counted by liquid scintillation. Assays were performed on three independent isolates of each genotype. Statistical analysis was performed using t-tests.

Lipid Accumulation

Assessment of the steady-state accumulation of lipids was performed by labeling a dilute inoculation (˜1/1000 of a saturated culture) of each strain into 10 mL of SC-His+2% galactose+1% raffinose with 0.01 μCi/mL of [³H] or [¹⁴C]oleate or 0.25 μCi/mL of [³H]palmitate and overnight growth at 30° C. Cells were washed and lipids were extracted and analyzed as described above.

In Vitro Assay of DGAT and AWAT Activity and Substrate Specificity in Yeast Microsomes

A dilute inoculation of each strain into 500 mL of SC-His+2% galactose+1% raffinose was grown overnight at 30° C. into log phase. Cells were washed and lysed, and microsomes were prepared from a 100,000 g spin, as previously described (39). Protein concentrations were determined (40) and diacylglycerol esterification assays were performed as described previously (17,19,41). In brief, microsomes (100 μg) were preincubated for 5 min at 37° in 100 mM TRIS, pH 7.5 containing 0.25 M sucrose, 1 mM EDTA, 10 mM MgCl₂, 20 μM fatty acid free BSA, 160 μM dioleoylglycerol (in 5 μl acetone, final volume 200 μl). The reaction was initiated by the addition of [¹⁴C]oleoyl-CoA (10-20,000 dpm/nmol, 40 μM) and stopped after 10 min by the addition of 4 ml of chloroform:methanol (2:1 v/v). Carrier TAG (10 μg) and internal standard ([³H]triolein, 30,000 dpm) were added followed by 0.8 ml water to separate phases. The lower chloroform layer containing the lipids was removed, evaporated to dryness, resuspended in 50 μl chloroform and individual lipid classes separated on Varian chromatography paper using a hexane:diethyl ether:acetic acid (170:30:1 v/v) mobile phase. The spot corresponding to TG was cut out and counted.

Acyl-CoA wax alcohol acyltransferase (AWAT) assays were performed as above with the following exceptions: 200 μM cetyl alcohol (unlabeled or [¹⁴C]-labeled 10,000 dpm/nmol) was substituted for DAG, oleoyloleate was used as carrier, and reactions were incubated for 20 min in a final volume of 250 μl. After separation by TLC, the spot corresponding to wax ester was cut out and counted. For alcohol and acyl-CoA substrate specificity studies, microsomal preparations from DGA2 (20 μg) and hDC4 (10 μg) were used. Kill reactions and vector controls were included for all experiments. The various wax alcohols and acyl-Co A's were substituted for cetyl alcohol or oleoyl Co A at the concentrations specified above. Activity is presented as pmol/min/mg protein.

Determination of Tissue Expression of DGAT2 Gene Family

Nested PCR was performed on all members of the DGAT2 gene family (except MGAT3) using 2 ng human cDNA, 10×PCR Buffer, 1.75 mM MgCl₂, 2 μM dNTP's, 1.5 Units Taq polymerase, and 1 μM oligo primers (Table 4)-designed to amplify a portion of the gene of interest. The initial PCR was held at 94° C. for 5 min followed by 28-30 cycles of 94° C. for 30 s, 55° C. for 30 s and 72° C. for 1.5 min. The second PCR was conducted under similar conditions using 10% of the initial PCR product, 1 μM of internal oligo primers, and a 72° C. extension time ranging from 40 s-1 m for 28-30 cycles. Tissue expression for MGAT3 was determined using the above components held at 94° C. for 5 min followed by 35 cycles of 94° C. for 15 s, 53° C. for 1 min and 72° C. for 2 min. PCR products were resolved on a 1-2% agarose, 0.5 μg/ml ethidium bromide gel. Splice variants for hDC4 and MGAT1 were gel isolated, purified and sequenced. First strand cDNA synthesis was performed on mRNA from human adipose tissue (kindly provided by A. Ferrante, Columbia University, NY), with and without reverse transcriptase (SuperScriptII, Invitrogen), using 1 μg of RNA. 10% of the product was used as the template in the initial reaction of a nested PCR (as described above) designed to amplify a portion of each of the DGAT2 gene family members. Primers were similar to those utilized in experiments above except for the 2 ^{nd} reaction using MGAT2 and MGAT3 (see Table 4). Expression of the DGAT2 gene family in skin was determined via standard and nested PCR as described above, using 2 ng of human skin cDNA and primers specific to each gene (Table 4).

In situ hybridization was performed on freshly cut human scalp frozen sections using anti-sense probes for DGA2, hDC3, hDC4 and counterpart sense probe controls using standard procedures. Gene specific PCR primers pairs described in Table 4 were used to amplify templates from the pRS423-GP vector harboring each cDNA insert. The resulting PCR products (˜1 Kb for DGA2, and ˜0.5 Kb for hDC3 and hDC4) were ligated to the pCRII dual promoter vector (T7 and SP6) using the TA-cloning kit (Invitrogen). After sequencing to determine the orientation of the insert, the vector was linearized using either BamHI (T7, sense) or XhoI (SP6, antisense). Subsequently, in vitro transcription and DIG-labeling reactions were carried out following the manufacturer's instructions (Roche). Finally, the DIG-labeled RNA probes were ethanol precipitated, quantified, and tested for alkaline phosphatase activity on nitrocellulose membranes to determine the optimal concentration for both sense and anti-sense probes. For hybridization, the slides were fixed in 4% paraformaldehyde/1×PBS, washed in PBS, followed by acetylation using acetic anhydride and subsequent washing in PBS. After pre-hybridization in hybridization buffer (Roche) for 2 hours at 58° C., the probes were added and the slides incubated overnight at the same temperature in a humidified chamber. The next day, slides were washed at different stringencies of SSC, treated with RNase A, blocked in blocking buffer and then incubated in anti-DIG (1/500) overnight at 4° C. On the third day, the slides were washed and the signal detected using NBT/BCIP substrate. After 24 hours, the reaction was stopped and slides were dehydrated and mounted. Pictures were taken on a regular compound light microscope equipped with a camera.

Results

The Human DGAT2 Gene Family, Conserved Motifs and Evolutionary Relationships

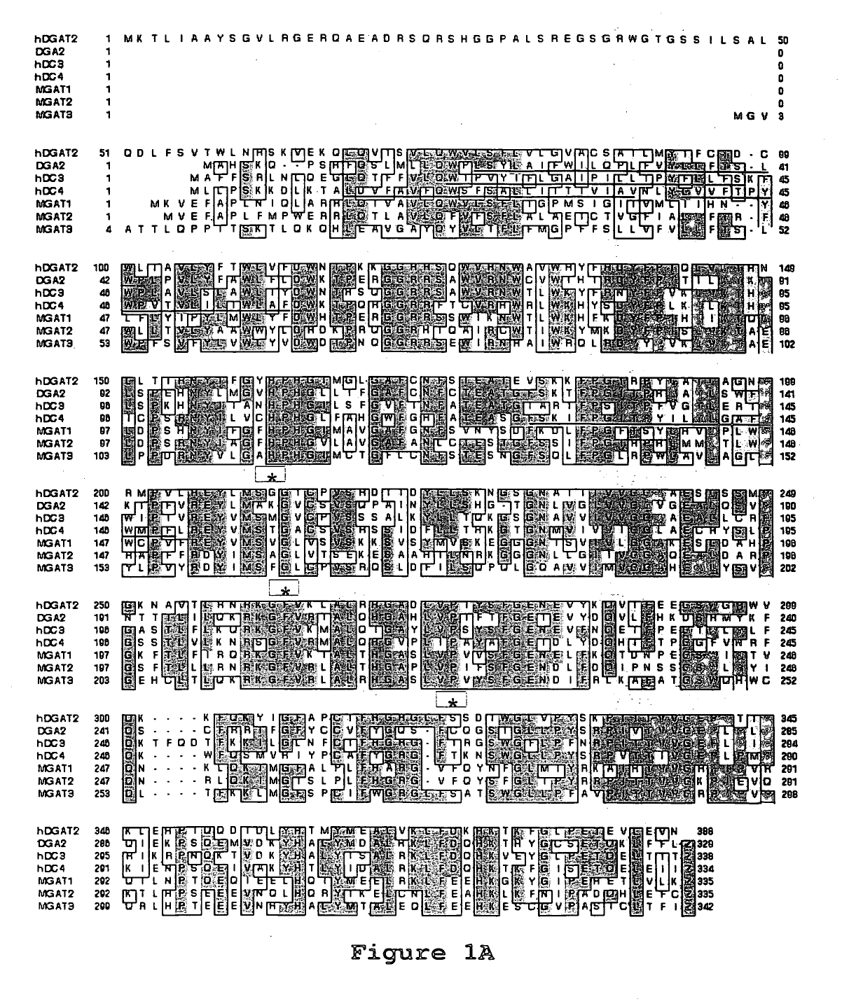

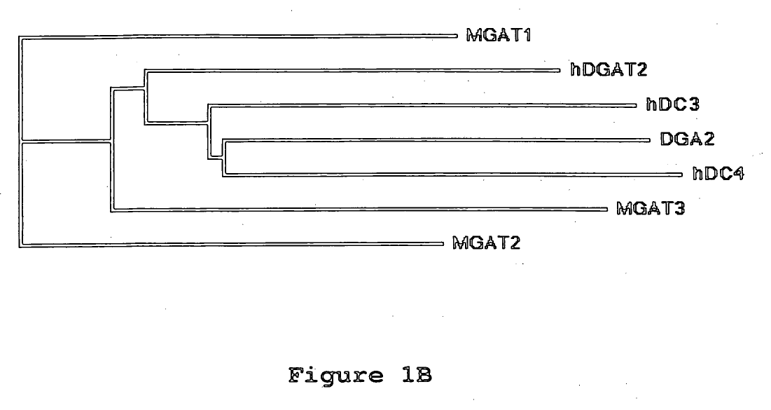

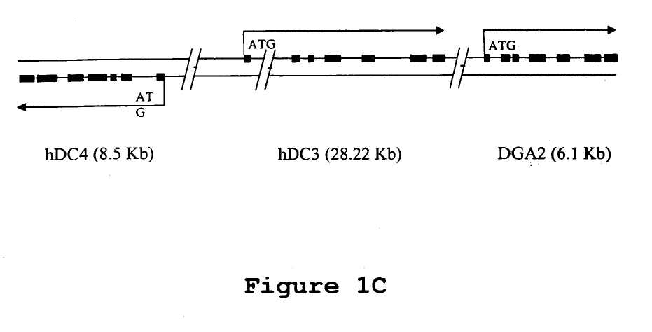

There are 6 paralogous genes to human DGAT2 whose chromosomal locations based on the human genome database are listed in Table 1. In addition to DGAT2 and MGATs 1-3, the previously termed hDC3 and hDC4 (human DGAT Candidate genes 3 & 4), and DGA2 (Diacyl-Glycerol Acyltransferases) were used as acronyms for the uncharacterized family members, upon the assumption that these genes direct the synthesis of triglyceride. The predicted polypeptides of these 6 genes exhibit 27-34% and 46-51% sequence identity to the primordial yeast DGA1 and human DGAT2 proteins, respectively, with marked conservation towards the carboxyl-terminus of the protein (FIG. 1A). All 7 human sequences and the yeast Dga1p are predicted transmembrane proteins (FIG. 1A). An iterative algorithm (PSI-BLAST, (30)), suggests these gene products to be derived from an ancestor with lysophosphatidic acid acyltransferase (LPAAT) or glycerol-3-phosphate acyltransferases (GPAT) activity. These enzymes and its relatives mediate the final steps in phosphatidic acid biosynthesis. This conservation includes those residues implicated by site directed mutagenesis as forming the active sites of bacterial GPAT (42).

The MGAT Arm of the DGAT2 Gene Family

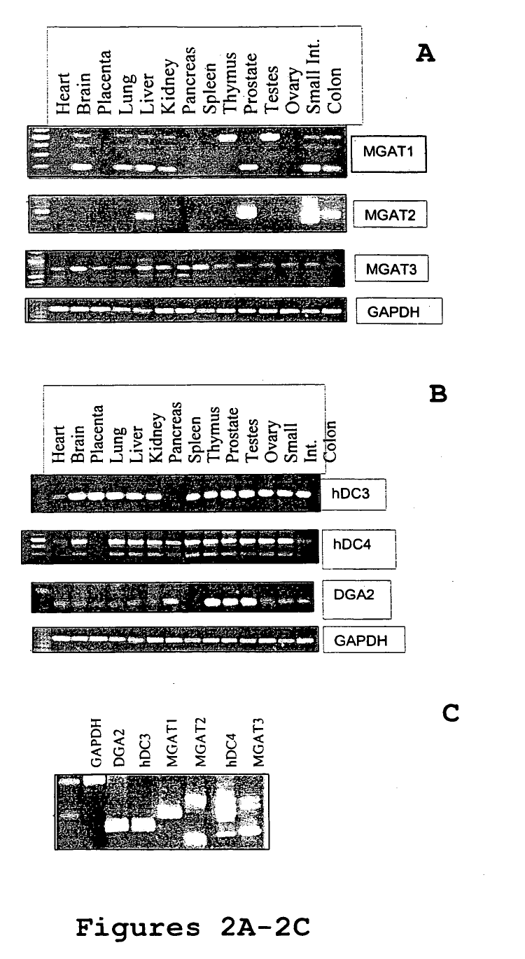

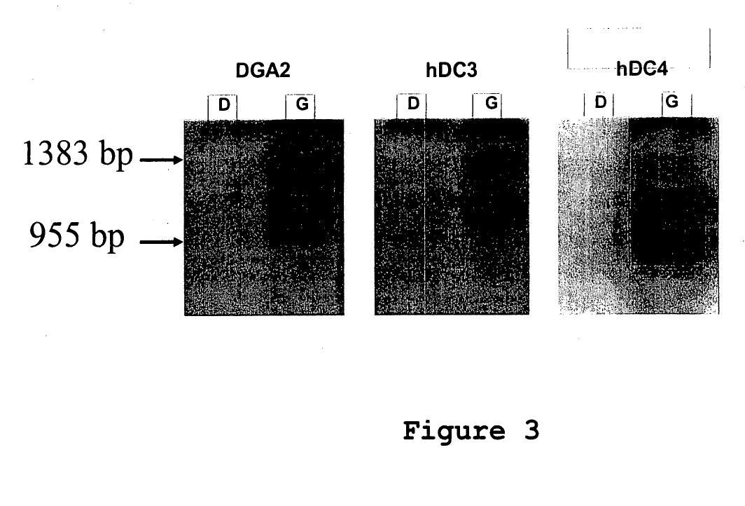

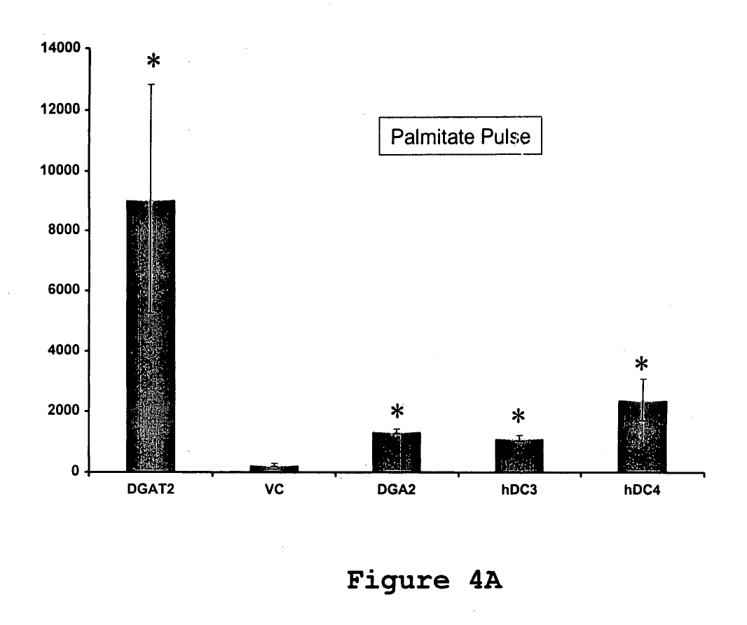

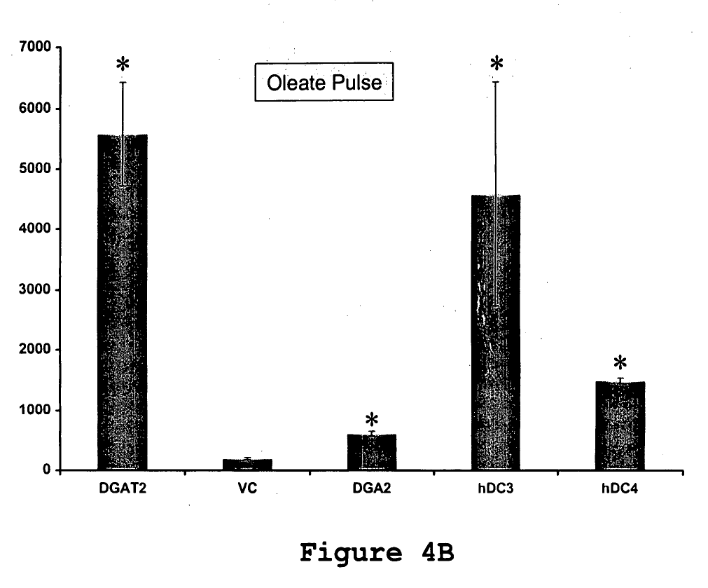

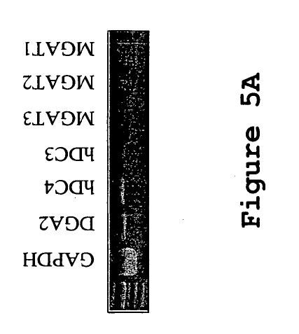

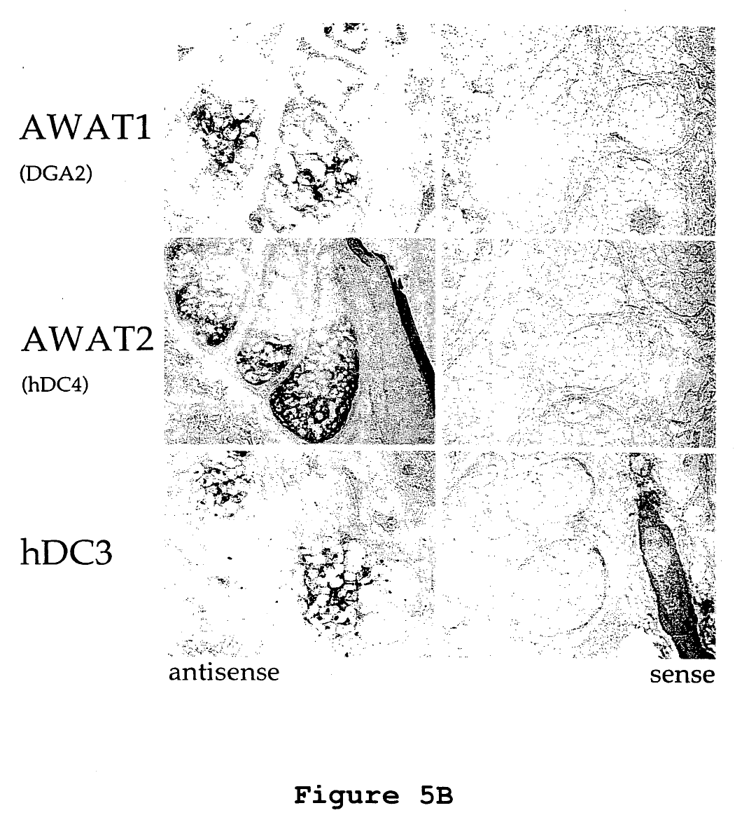

The MGAT branch of the DGAT2 gene family has been extensively characterized (21-26), primarily in murine tissues. Nested PCRs using cDNA were perfomed from a variety of tissues to confirm and further discern the tissue expression of human MGAT's 1-3 (FIG. 2A). Similar to mouse MGAT1, the human paralog is transcribed in stomach, uterus, kidney, adipose, and liver. However, unlike mice, human brain, lung, thymus, prostate, testes, colon and notably, small intestine had significant expression. Two splice variants of human MGAT1 exist, and the larger of the two is the predominant transcript found in thymus and testes. Based on sequence analysis, the larger transcript results from the splicing out of the third exon of MGAT1 and an alternatively spliced-in portion of intron 4; this introduces an early stop codon at base pair 312, truncating the protein. The smaller transcript is a product of exon 3 being spliced out, also predicting a truncated protein due to an early stop at base pair 294.

Human MGAT2 transcripts were identified in liver, prostate, small intestine, colon and adipose (FIG. 2A, C), confirming previous findings (22,26). In contrast to mMGAT2 (24,25) and a previous report on hMGAT2 (22), no expression was found in kidney tissue. Consistent with previous reports (22,26), splice variants were also exhibited by MGAT2, especially in adipose tissue.

Extensive searching of the available murine databases indicates that the MGAT3 gene is not present in mice. It has been previously reported that hMGAT3 expression is restricted to the gastrointestinal tract and the liver (23). In the instant study, (FIG. 2A) the full-length hMGAT3 is found in liver, small intestine and colon. However, splice variants of hMGAT3 are ubiquitously found in many human tissues. The larger of these splice variants results from the splicing out of exon 5, leading to a truncated protein due to the creation of an early stop codon at base pair 774.

The functional significance of the human MGAT1, MGAT2 and MGAT3 splice variants remains to be determined. MGAT activity has been considered a reaction exclusive to the intestine, at least in rodents (43). The expression patterns of this branch of the human DGAT2 family are inconsistent with this concept. However, if the aforementioned conservation with the LPAAT enzymes is significant, then the majority of these splice variants would be anticipated to be inactive due to premature truncation. This may implicate these proteins as having alternate activities or regulatory roles in neutral lipid metabolism. For example, why is the larger MGAT1 message the sole transcript found in thymus and testes? Interestingly, in humans, all of the MGATs, including the full-length transcripts, are expressed in adipose tissue (FIG. 2C).

DGAT2 and the Sex-Linked Arm of the DGAT2 Gene Family

Human and murine DGAT2 have been characterized extensively (9,10). By contrast the X-linked hDC4-hDC3-DGA2 cluster (Xq13.1) of this gene family has not been investigated previously with regard to function or expression. Efforts were focused on defining the role of this sub-family in neutral lipid biosynthesis. DGA2 is previously undescribed and comprises 7 exons covering 6.1 kb on the direct strand, approximately 190 kb downstream from hDC4 and 29 kb downstream from hDC3. It encodes a 329-amino acid protein with 51% amino acid identity to hDGAT2 and a predicted molecular weight of 37.9 kDa. DGA2 possesses the diacylglycerol acyltransferase (DAGAT) motif common to this gene family and has one predicted transmembrane region (amino acids 21-42), one potential N-glycosylation site and one potential protein kinase C phosphorylation site. The initial 44 amino acids of DGA2 are predicted to be a signal peptide. Interestingly, DGA2 possesses a region with remote similarity to the IIGF (Insulin-like and Insulin Growth Factor) domain, which belongs to a family of proteins including insulin and insulin-like growth factors.

hDC4 (10) encodes a 334-amino acid protein with similarity to the phosphate acyltransferase domain of GPAT, LPAAT, and other related acyltransferases. hDC4 has a predicted molecular mass of 38.2 kDa, and is 48% identical to hDGAT2 and 51% identical to DGA2 (Table 1). hDC4 has three predicted transmembrane regions (amino acids 13-35, 39-58 & 127-149) as well as one potential N-glycosylation site, one potential tyrosine sulfation site, and two potential protein kinase C phosphorylation sites. The initial 35 amino acids of hDC4 are predicted to be a signal peptide. hDC4 covers 8.5 kb on the reverse strand of the X chromosome and comprises 7 exons.

hDC3 (10) encodes a 338-amino acid protein also with a phosphate acyltransferase domain similar to the LPAAT family. hDC3 is 50% identical to hDGAT2, 51% identical to hDC4, and 51% identical to DGA2. It has a predicted molecular mass of 38.7 kDa and possesses at least one transmembrane region (amino acids 21-43) in addition to one potential N-glycosylation site and three potential protein kinase C phosphorylation sites. hDC4 is comprised of 7 exons covering 28.22 kb on the direct strand of the X chromosome, approximately 130 kb downstream from the DGA3 gene.

To examine the expression of DGA2, hDC3 and hDC4 in humans, nested PCR was performed using cDNA generated from a variety of tissues (FIG. 2B). DGA2 is expressed in all tissues except the spleen; interestingly, the strongest bands were found in thymus, prostate, and testes. hDC4 is expressed in all tissues surveyed except the placenta whereas hDC3 is expressed in all tissues except pancreas. Based on automated DNA sequencing, it was determined that a splice variant of hDC4 results from the excision of the 175 base pair exon 5. This creates an early stop codon at base pair 483 in the resultant transcript. The physiological function of this splice variant remains to be determined. All three genes are expressed in human adipose tissue (FIG. 2C).

Expression of DGAT2 in Yeast Cells Deficient in TG Synthesis

To examine the expression and biochemical activity of DGAT2, its cDNA was expressed in a diacylglycerol esterification deficient yeast strain (SCY2056, (17)) in which the endogenous DGA1, LRO1 and ARE2 genes were deleted. Metabolic labeling experiments performed with [³H]/[¹⁴C]oleate and [³H]palmitate are consistent with a major role of DGAT2 in triglyceride synthesis (FIG. 4) and to a lesser extent, diacylglycerol synthesis (not shown) (21). It has been previously shown that yeast deficient in triglyceride and steryl ester synthesis display minimal cytoplasmic lipid droplets when stained with the vital stain Nile Red and followed by fluorescence microscopy (17). Upon expression of human DGAT2, these strains exhibit a marked accumulation of cytoplasmic neutral lipid droplets (not shown). In addition, microsomes from null TAG background strains of yeast, transformed with an expression vector harboring no insert or the cDNA insert for DGAT2 were assayed in vitro for the incorporation of [¹⁴C]oleate and DAG into triglyceride. As shown in Table 2, in an in vitro microsomal assay, DGAT2 forms triglyceride at a rate of 65.4 pmol/min/mg protein.

Expression of the Sex-Linked Arm of the DGAT2 Gene Family in Yeast

The previous experiments with human DGAT2 demonstrate the utility of this heterologous system in the analysis of mammalian triglyceride synthesis as was demonstrated-previously for sterol esterification by members of the ACAT gene family (35,44). Similar investigations were performed expressing the cDNA of DGA2, hDC3, and hDC4 in the null TAG background strains of yeast. Northern blot hybridization analysis confirmed the high level expression of DGA2, hDC3 and hDC4 transcripts of the predicted size (FIG. 3).

During pulse and steady state [³H]/[¹⁴C]oleate and [³H]palmitate metabolic labeling, DGA2, hDC3, and hDC4 exhibit significant triglyceride production, above background strains (FIG. 4). Microsomes from null TAG background strains of yeast, transformed with an expression vector containing DGA2, hDC3 and hDC4 were assayed in vitro for the incorporation of [¹⁴C] oleate and DAG into triglyceride. In each case significant synthesis of TAG was detected (Table 2).

Substrate Specificity of Sex-Linked Arm of the DGAT2 Gene Family

Although DGA2, hDC3 and hDC4 were capable of synthesizing TG in transformed yeast (FIG. 4) and demonstrate in vitro DGAT activity (Table 2), the low specific activity, particularly compared to DGAT2, suggests that DAG is not their primary substrate. No increase in esterification of MAG or glycerol phosphate above vector control was detected (not shown), leading to a search for other alcohols as substrates. Recently a novel bifunctional Wax Ester Synthase/Acyl-CoA:Diacylglycerol Acyltransferase that shares no detectable similarity with the DGAT2 gene family was discovered in Acinetobacter calcoaceticus (45). This, in combination with the fact that a mammalian acyl-CoA wax alcohol acyltransferase (AWAT) sequence was unknown at the time of this study, led to the assay of DGA2, hDC3 and hDC4 strains for this activity. Initial experiments were performed using cetyl alcohol (C16) and oleoyl CoA as substrates.

Both DGA2 (31.7 pmol/min/mg) and hDC4 (1066 pmol/min/mg) but not DGAT2 or hDC3 demonstrated significant AWAT activity using [¹⁴C]oleoyl CoA as a monitor for wax ester formation (Table 2). Wax esters co-migrate with sterol esters on thin layer chromatography. Although the vector control contained one of the two yeast enzymes responsible for sterol esterification (ARE1), it is unlikely that the activity observed is due to sterol ester formation because yeast sterols are not preferred substrates for the mammalian enzymes (46,47). However, to verify this, an identical assay was performed using [¹⁴C]cetyl alcohol rather than radio labeled oleoyl CoA as a monitor of ester formation. Again, significant AWAT activity was observed with DGA2 (113 pmol/min/mg) and hDC4 (1232 pmol/min/mg) demonstrating that wax ester, not sterol ester, is the enzymatic product and leading to the renaming of these proteins as AWAT1 and AWAT2, respectively.

The substrate specificities of AWAT1 and AWAT2 were then investigated in detail. As shown (Table 3A) the alcohol substrate specificities of the two enzymes are strikingly dissimilar. Using oleoyl CoA as the acyl donor, AWAT1 (DGA2) has a definite preference for decyl alcohol (C10), with less activity using C16 and C18 unsaturated alcohols. AWAT1 utilizes arachidyl alcohol about 5% as well as decyl alcohol, demonstrating its relatively poor activity using saturated long chain alcohols (C16, C18 and C20). In contrast, AWAT2 exhibited no activity using decyl alcohol and significantly preferred the C16 and C18 alcohols.

AWAT1 and AWAT2 also showed a difference in acyl-CoA preference (Table 3B). Using cetyl alcohol as the acyl acceptor, AWAT1 shows a strong preference towards the saturated acyl group; it uses oleoyl CoA (C18:1) only 20% as well as stearoyl CoA (C18:0) and has no activity using palmitoleoyl CoA (C-16:1). In contrast, AWAT2 demonstrates significant activity using all four acyl-CoAs and utilizes unsaturated acyl-CoAs twice as well as saturated acyl-CoAs under the conditions employed.

Expression of AWAT1, AWAT2 and hDC3 in Human Skin

In addition to several other neutral lipids such as TAG, wax esters are major components of sebum, a production of the sebaceous gland. Therefore, human skin cDNA preparations were tested for the expression of these genes and discovered that transcripts from all 7 members of the DGAT2 family were detectable, with particularly high levels of expression of the X-linked sub-family (FIG. 5A). To identify the cell types involved, in situ hybridizations of human skin sections were performed with sense and antisense probes for AWAT1, AWAT2 and hDC3 (FIG. 5B). Control sense probes did not result in section staining. Expression of AWAT1 and AWAT2 was clearly limited to the sebaceous gland, with AWAT2 primarily restricted to the cytoplasm of undifferentiated peripheral sebocytes. AWAT1 was expressed in more mature, centrally located cells just before their rupture and sebum release. hDC3 transcripts were also localized to the cells of the sebaceous gland, however the predominant staining corresponded to the nuclei, raising the possibility that the gene is transcribed but not translated in sebocytes.

Discussion

Neutral lipids such as TAG and SE are commonly sequestered in the core of cytoplasmic lipid droplets until such time when hydrolysis allows their return to the metabolic fray. This efficient process of energy storage and release is widely used in nature to facilitate such diverse processes as hibernation in animals, embryo development in eggs and plant seeds and perhaps sporulation in yeast. Neutral lipid biosynthesis therefore represents an advantageous response to overabundance and thus potential toxicity of membrane lipids and energy. Given this pivotal role in cellular metabolism, it is perhaps not surprising to discover so many distinct pathways (PDAT, DGAT1 and DGAT2 mediated, to date) and genes for the production of TG. The complexity of the large gene family associated with the DGAT2 reaction is perhaps more surprising and likely reflects alternate substrate specificities. For example, although MGATs 1-3 appear to be genealogically closely related to DGAT2, they differ markedly in their substrate specificity and expression patterns. Similarly, it is demonstrated here that the previously uncharacterized X-linked members of this family have only modest DGAT activity in vivo and in vitro. Instead, it is demonstrated that two members of this sub-division of the DGAT2 gene family are responsible for the generation of acyl esters of long chain alcohols (fatty alcohols) in the synthesis of wax esters. Interestingly, plant orthologs of DGAT2 have been reported to mediate the synthesis of both TG and wax esters (48). Recently, the murine ortholog of AWAT2 has been demonstrated to also perform this reaction (49). Based on sequence conservation, the three X-linked members of this 7-component family are closer to DGAT2 than to the MGAT sub-family (FIG. 1B). Thus it is possible that the final member of this gene family, hDC3 is a bone-fide DGAT enzyme. The enzyme was expressed in yeast and clearly conferred the ability to synthesize significant levels of triacylglycerol. Alternatively, hDC3 may be an orphan waiting for an activity and substrate to be ascribed. Interestingly, the hDC3 mRNA appears to remain within the nucleus in skin sections and in situ hybridizations.

The chromosomal location of these genes may implicate them as candidates for several human syndromes ranging from obesity to skin disorders. For example, the hDC3, hDC4 and DGA2 genes comprise a cluster of 3 physically linked genes localizing to the X chromosome, in a contig of approximately 200 Kbp, in mice and humans (FIG. 1C). Interestingly there are several undefined sex-linked obesity (e.g. Borjeson-Forssman-Lehmann and Wilson-Turner syndromes, see (50) for review) and dermatological syndromes (51). Similarly, it is interesting to note that chromosome 11 (specifically 11q13) containing DGAT2, MGAT2, uncoupling protein UCP2 and UCP3 (52,53), and BBS1, the gene most commonly involved in Bardet-Biedl syndrome (54), has been linked to obesity and hyper-insulinaemia. Similarly, one genome scan also implicated the region of 11q13 in childhood and adolescent obesity (55). Moreover, several linkage studies suggest that 7q22.1, a region flanking the leptin gene that also maps to MGAT3, is closely linked to obesity and body mass index in humans (56,57).

In addition to their roles as safe harbors for toxic fatty acids or alternative form of energy storage, molecules such as wax esters and TAG function as a hydrophobic permeability barrier to limit dehydration from tissue surfaces (58,59). As such, both molecules are significant components of plant cuticle (60), insect exoskeleton coating (61), and mammalian sebum. Indeed, murine DGAT1 and DGAT2 utilize long chain alcohols, in addition to DAG, as substrates (49), and are critical for epidermal integrity (12,15). Wax esters are also the main component of spermaceti that allows for sperm whales to regulate their buoyancy. While the many commercial applications of whale oil prompted sperm whale hunting, purification of wax esters from the jojoba plant currently meets the market's demands for wax esters. The AWAT reaction thus has both commercial and biological relevance.

Until recently ((49) and this study), the AWAT reaction was uncharacterized at the molecular level. A novel bifunctional Wax Ester Synthase/Acyl-CoA:Diacylglycerol Acyltransferase that shares no detectable similarity with the DGAT2 gene family and has no human counterpart was discovered in Acinetobacter calcoaceticus (45). Thus, murine wax ester synthase (49), human AWAT1, and human AWAT2 likely represent the most significant contributors to the pool of wax esters in sebum in mammals. Indeed, the tissue and cell type expression pattern for these genes is consistent with a major role for these enzymes in the secretion of sebum by the sebocyte. The most common saturated fatty acids found in sebum wax esters are C16>C14>C15 whereas palmitoleoyl (C16:1) acid is the most abundant mono-unsaturated fatty acid being present at 5 times the amount of oleic acid (62). Interestingly AWAT1 shows no activity towards palmitoleoyl CoA while AWAT2 and its murine ortholog (49) prefer unsaturated fatty acids. The diet appears to have little effect on wax ester composition with linoleic acid, an exclusively dietary fatty acid accounting for <1% of wax ester fatty acids (63). This would explain why disruption of stearoyl CoA desaturase (responsible for synthesis of palmitoleic and oleic acids) causes atrophy of sebaceous gland (64).

Strikingly, AWAT1 and 2 are expressed at different stages in the differentiation of sebocytes (FIG. 5B), suggesting a temporal reason for quality control of the wax esters. It is possible that as the sebocyte matures, some form of wax ester hydrolysis and re-esterification occur, such that the wax esters are remodeled prior to their secretion.

Tables

TABLE 1
The human DGAT2 gene superfamily

| Gene | Chromosome | Amino Acids | % Identity to DGAT2 |
|---|---|---|---|
| hDGAT2 | 11q13.5 | 388 | 100 |
| DGA2 | Xq13.1 | 329 | 51 |
| hDC3 | Xq13.1 | 338 | 50 |
| hDC4 | Xq13.1 | 334 | 48 |
| MGAT1 | 2q36.3 | 335 | 50 |
| MGAT2 | 11q13.5 | 335 | 46 |
| MGAT3 | 7q22.1 | 342 | 49 |

Sequences were derived and compared by BLAST homology searching of the human genomes databases (NCBI, Ensembl, UCSC & Celera). Chromosomal assignments are as described. DGAT2, hDC3, and hDC4 were initially described by Cases et al (10). Two groups have ascribed monoacylglycerol acyltransferase activity to MGAT1, MGAT2 (21,22) and MGAT3 (23). DGA2 (DiacylGlycerol Acytransferase 2) is described in the current study.

TABLE 2
In vitro Acyltransferase assays

| Nomenclature | DGAT Activity | AWAT Activity |
|---|---|---|
| DGAT2 | 65.4 +/− 3.4 | −11.5 +/− 1.3 |
| DGA2 | 7.8 +/− 3.2 | 31.7 +/− 17.4 |
| hDC3 | 13.3 +/− 4.6 | −25.7 +/− 9.3 |
| hDC4 | 11.4 +/− 3.4 | 1066 +/− 18.6 |

Microsomes from TG-null yeast strains transformed with human DGAT2, DGA2, hDC3, or hDC4 were incubated in the presence of dioleoylglycerol or cetyl alcohol and [¹⁴C]oleoyl-CoA and assayed for esterification as described in Experimental Procedures. Data are expressed as pmol of neutral lipid (TAG or Wax ester) formed per min/mg protein as a mean±Std. dev. DGAT activities are from 3 replicate experiments and AWAT activities are from a representative experiment performed in triplicate. Background levels to the assay from strains transformed with vector control have been subtracted. All DGAT activity values and AWAT activity from DGA2 and hDC4 were significantly elevated relative to the vector control (t-test comparison).

TABLE 3A
Activity of AWAT1 and AWAT2
Towards Various Wax Alcohols

| Alcohol | AWAT1 (DGA2) | AWAT2 (hDC4) |
|---|---|---|
| Decyl C-10 | 419 +/− 180 | −50 +/− 277 |
| Cetyl (Palmityl) C-16 | 83 +/− 6 | 665 +/− 54 |
| Palmitoleyl C-16:1 | 184 +/− 29 | 582 +/− 117 |
| Stearyl C-18 | 27 +/− 5 | 138 +/− 24 |
| Oleyl C-18:1 | 247 +/− 23 | 892 +/− 106 |
| Arachidyl C-20 | 22 +/− 4 | 27 +/− 4 |

TABLE 3B
Activity of AWAT1 and AWAT2
Towards Various Acyl - CoAs

| Acyl - CoA | AWAT1 (DGA2) | AWAT2 (hDC4) |
|---|---|---|
| Palmitoyl C-16 | 390 +/− 121 | 524 +/− 164 |
| Palmitoleoyl C-16:1 | −20 +/− 25 | 1125 +/− 220 |
| Stearoyl C-18 | 656 +/− 39 | 549 +/− 38 |
| Oleoyl C-18:1 | 113 +/− 7 | 1232 +/− 349 |

Substrate Specificities of Acyl-CoA Wax Alcohol Transferases (AWAT) 1 and 2

In vitro AWAT assays were performed using microsomal preparations from TG null yeast strains transformed with human AWAT1 (DGA2), AWAT2 (hDC4) or a vector control harboring no insert. AWAT1 (20 μg) and AWAT2 (10 μg) microsomes were assayed as described under Experimental Procedures. Activity is in pmol of wax ester formed per min/mg protein, expressed as a mean±Std. dev. from a representative experiment performed in triplicate. Vector control background has been removed. In vitro determination of alcohol and acyl-CoA substrate specificities were performed with [¹⁴C]oleoyl-CoA and the indicated alcohols or [¹⁴C]cetyl alcohol and the indicated acyl-CoA donors (Table 3A and 3B, respectively). Kill reactions were included for all experiments.

TABLE 4

Oligonucleotides for expression,
cloning and in-situ hybridization

| | | | | |
|---|---|---|---|---|
| Gene | Reaction | Purpose | 5′ Forward Primer | 3′ Reverse Primer |

| | | | | |
|---|---|---|---|---|
| DGA2 | Reaction #1 | Tissue | GAATTCATGGCTCATTCCAAGCAGCCTA | GTCTCTGAGCAGCCATAGTG |
| | Reaction #2 | Expression | ATGGCTCATTCCAAGCAGCCTA | CGTAATGGGGAAATAGTCCCT |
| | Reaction #1 | Tissue | TTGCCTCCCTCAGGCTCCCGAGAATC | CCTGGGCATGCAGTACCTTCAT |
| | Reaction #2 | Expression | GAATTCATGGCTCATTCCAAGCAGCCTA | GCGGCCGCTCACAGGAAAAACAGCTTTTGGG |
| | Reaction #1 | TA cloning | ATGGCTCATTCCAAGCAGCCTA | GTCTCTGAGCAGCCATAGTG |
| | Reaction #2 | TA cloning | GAATTCATGGCTCATTCCAAGCAGCCTA | GTCTCTGAGCAGCCATAGTG |
| | | In-situ probe | ATGGCTCATTCCAAGCAGCCTA | CGTAATGGGGAAATAGTCCCT |
| | | Skin Expression | | |
| | | Skin Expression | | |
| hDC3 | Reaction #1 | Tissue | ATGGCTTTCTTCTCCCGACTGAAT | CACTGATGTAGAGTGCGTGA |
| | Reaction #2 | Expression | CTGGTGAAGACTCATGATCT | TCAAGGCTGAGCTACTCACA |
| | Reaction #1 | Tissue | ATGGCTTTCTTCTCCCGACTGAAT | TCAAGGCTGAGCTACTCACA |
| | Reaction #2 | Expression | ATGGCTTTCTTCTCCCGACTGAAT | CACTGATGTAGAGTGCGTGA |
| | | In-situ probe | CTGGTGAAGACTCATGATCT | TCAAGGCTGAGCTACTCACA |
| | | Skin Expression | | |
| | | Skin Expression | | |
| hDC4 | Reaction #1 | Tissue | GAATTCATGCTCTTGCCCTCTAAGAAGGA | GCGGCCGCTCAAATTATCTCCAGCTCCTGG |
| | Reaction #2 | Expression | TCTGAAGACTCATGACATCT | ATGTGCTGATCATAGAGGTC |
| | Reaction #1 | Tissue | AGCCACTGGGACGCTGGGCACA | TGAAGGCTACTGGGGATGTCTG |
| | Reaction #2 | Expression | GAATTCATGCTCTTGCCCTCTAAGAAGGA | GCGGCCGCTCAAATTATCTCCAGCTCCTGG |
| | Reaction #1 | TA cloning | TCTGAAGACTCATGACATCT | ATGTGCTGATCATAGAGGTC |
| | Reaction #2 | TA cloning | GAATTCATGCTCTTGCCCTCTAAGAAGGA | GCGGCCGCTCAAATTATCTCCAGCTCCTGG |
| | | In-situ probe | TCTGAAGACTCATGACATCT | ATGTGCTGATCATAGAGGTC |
| | | Skin Expression | | |
| | | Skin Expression | | |
| MGAT1 | Reaction #1 | Tissue | ATGAAGGTAGAGTTTGCACC | TCATTTTAAAACAAGAGTCT |
| | Reaction #2 | Expression | CTGTGATGCTGATCATACAC | AGAGTGAACTTTCCAGGATG |
| | Reaction #1 | Tissue | ATGAAGGTAGAGTTTGCACC | TCATTTTAAAACAAGAGTCT |
| | Reaction #2 | Expression | CTGTGATGCTGATCATACAC | AGAGTGAACTTTCCAGGATG |
| | | Skin Expression | | |
| | | Skin Expression | | |
| MGAT2 | Reaction #1 | Tissue | GAATTCATGGTAGAGTTCGCGCCCTTGT | GCGGCCGCTCAGCAGAACTCCAAGTGCTG |
| | Reaction #2 | Expression | TGCAGACACTTGCTGTCCTA | ATGCAGCGTCTTCTGTACCT |
| | Reaction #2 | Tissue | TGCAGACACTTGCTGTCCTA | CAGAGATTACATCATGTCTGC |
| | Reaction #1 | Expression | GAATTCATGGTAGAGTTCGCGCCCTTGT | GCGGCCGCTCAGCAGAACTCCAAGTGCTG |
| | Reaction #2 | Expression | TGCAGACACTTGCTGTCCTA | ATGCAGCGTCTTCTGTACCT |
| | | (adipose) | | |
| | | Skin Expression | | |
| | | Skin Expression | | |
| MGAT3 | Reaction #1 | Tissue | CTTGCAGAAGCAGCATCTAG | GCGTGATAGTGATTGACTT |
| | Reaction #1 | Expression | ATGGGAGTTGCCACAACCCTGCAG | GCGTGATAGTGATTGACTT |
| | Reaction #2 | Expression | CTTGCAGAAGCAGCATCTAG | CATGATGTAGTCGCGATAGA |
| | Reaction #1 | (adipose) | ATGGGAGTTGCCACAACCCTGCAG | GCGTGATAGTGATTGACTT |
| | Reaction #2 | Expression | CTTGCAGAAGCAGCATCTAG | CATGATGTAGTCGCGATAGA |
| | | (adipose) | | |
| | | Skin Expression | | |
| | | Skin Expression | | |

Primer sequences employed for DGAT2 gene family tissue expression, TA cloning of DGA2 and hDC4, and creation of probes for in-situ hybridization of human skin sections with DGA2, hDC3 and hDC4.

### REFERENCES

1. Oelkers, P. M., and Sturley, S. L. (2004) in Lipid metabolism and membrane biogenesis (Daum, G., ed) Vol. 6, pp. 281-311, Springer-Verlag, Berlin
2. Yu, Y. H., and Ginsberg, H. N. (2004) Ann Med 36, 252-261
3. Coleman, R. A., Lewin, T. M., and Muoio, D. M. (2000) Annu Rev Nutr 20, 77-103
4. Subauste, A., and Burant, C. F. (2003) Curr Drug Targets Immune Endocr Metabol Disord 3, 263-270
5. Ross, R. (1995) in Molecular Cardiovascular Medicine (Haber, E., ed), pp. 11-30, Scientific American, New York
6. Krauss, R. M. (1998) Am J Med 105, 58S-62S
7. Mulhall, B. P., Ong, J. P., and Younossi, Z. M. (2002) J Gastroenterol Hepatol 17, 1136-1143
8. Cases, S., Smith, S. J., Zheng, Y. W., Myers, H. M., Lear, S. R., Sande, E., Novak, S., Collins, C., Welch, C. B., Lusis, A. J., Erickson, S. K., and Farese, R. V., Jr. (1998) Proc Natl Acad Sci USA 95, 13018-13023
9. Lardizabal, K. D., Mai, J. T., Wagner, N. W., Wyrick, A., Voelker, T., and Hawkins, D. J. (2001) J Biol Chem 276, 38862-38869
10. Cases, S., Stone, S. J., Zhou, P., Yen, E., Tow, B., Lardizabal, K. D., Voelker, T., and Farese, R. V., Jr. (2001) J Biol Chem 276, 38870-38876
11. Smith, S. J., Cases, S., Jensen, D. R., Chen, H. C., Sande, E., Tow, B., Sanan, D. A., Raber, J., Eckel, R. H., and Farese, R. V., Jr. (2000) Nat Genet 25, 87-90
12. Chen, H. C., Smith, S., Tow, B., Elias, P., and Farese, R. J. (2002) J Clin Invest. 109, 175-181
13. Chen, H., Smith, S., Ladha, Z., Jensen, D., Ferreira, L., Pulawa, L., McGuire, J., Pitas, R., Eckel, R., and Farese, R. J. (2002) J Clin Invest. 109, 1049-1055
14. Chen, H., Ladha, Z., and Farese, R. J. (2002) Endocrinology 143, 2893-2898
15. Stone, S. J., Myers, H. M., Watkins, S. M., Brown, B. E., Feingold, K. R., Elias, P. M., and Farese, R. V., Jr. (2003) J Biol Chem
16. Sandager, L., Dahlqvist, A., Banas, A., Stahl, U., Lenman, M., Gustavsson, M., and Stymne, S. (2000) Biochem Soc Trans 28, 700-702
17. Oelkers, P., Cromley, D., Padamsee, M., Billheimer, J. T., and Sturley, S. L. (2002) J Biol Chem 277, 8877-8881
18. Sorger, D., and Daum, G. (2002) J Bacteriol 184, 519-524
19. Oelkers, P., Tinkelenberg, A., Erdeniz, N., Cromley, D., Billheimer, J. T., and Sturley, S. L. (2000) J Biol Chem 275, 15609-15612
20. Dahlqvist, A., Stahl, U., Lenman, M., Banas, A., Lee, M., Sandager, L., Ronne, H., and Stymne, S. (2000) Proc Natl Acad Sci USA 97, 6487-6492
21. Yen, C. L., Stone, S. J., Cases, S., Zhou, P., and Farese, R. V., Jr. (2002) Proc Natl Acad Sci USA 99, 8512-8517
22. Yen, C. L., and Farese, R. V., Jr. (2003) J Biol Chem 278, 18532-18537
23. Cheng, D., Nelson, T. C., Chen, J., Walker, S. G., Wardwell-Swanson, J., Meegalla, R., Taub, R., Billheimer, J. T., Ramaker, M., and Feder, J. N. (2003) J Biol Chem 278, 13611-13614
24. Cao, J., Burn, P., and Shi, Y. (2003) J Biol Chem
25. Cao, J., Lockwood, J., Burn, P., and Shi, Y. (2003) J Biol Chem 278, 13860-13866
26. Lockwood, J., Cao, J., Burn, P., and Shi, Y. (2003) Am J Physiol Endocrinol Metab, Epub ahead of print
27. Ausubel, F. M., Brent, R., Kingston, R. E., Moore, D. D., Seidman, J. G., Smith, J. A., and Struhl, K. (1998) Current Protocols in Molecular Biology, John Wiley & Sons, New York
28. Maniatis, T., Fritsch, E. F., and Sambrook, J. (1987) Molecular Cloning: A Laboratory Manual, Cold Spring Harbor Lab., Cold Spring Harbor, N.Y.
29. Altschul, S. F., Gish, W., Miller, W., Myers, E. W., and Lipman, D. J. (1990) J. Mol. Biol. 215, 403-410
30. Altschul, S. F., Madden, T. L., Schaffer, A. A., Zhang, J., Zhang, Z., Miller, W., and Lipman, D. J. (1997) Nucleic Acids Res. 25, 3389-3402
31. Marck, C. (1988) Nucl Acids Res 16, 1829-1836
32. Thompson, J. D., Higgins, D. G., and Gibson, T. J. (1994) Nucleic Acids Res 22, 4673-4680
33. Bairoch A., Bucher P., and K., H. (1997) Nucleic Acids Res. 25, 217-221
34. Letunic, I., Copley, R. R., Schmidt, S., Ciccarelli, F. D., Doerks, T., Schultz, J., Ponting, C. P., and Bork, P. (2004) Nucleic Acids Res 32 Database issue, D142-144
35. Yang, H., Cromley, D., Wang, H., Billheimer, J. T., and Sturley, S. L. (1997) J Biol Chem 272, 3980-3985
36. Ito, H., Fukuda, Y., Murata, K., and Kimura, A. (1983) J. Bacteriol. 153, 163-168
37. Tinkelenberg, A. H., Liu, Y., Alcantara, F., Khan, S., Guo, Z., Bard, M., and Sturley, S. L. (2000) J. Biol. Chem. 275, 40667-40670
38. Freeman, C. P., and West, D. (1966) J Lipid Res 7, 324-327
39. Zinser, E., and Daum, G. (1995) Yeast 11, 493-536
40. Lowry, O. H., Rosebrough, N. J., Farr, A. L., and Randall, R. J. (1951) J. Biol. Chem. 193, 265-275
41. Rustan, A. C., Nossen, J. O., Christiansen, E. N., and Drevon, C. A. (1988) J Lipid Res 29, 1417-1426
42. Lewin, T. M., Wang, P., and Coleman, R. A. (1999) Biochemistry 38, 5764-5771
43. Lehner, R., and Kuksis, A. (1992) Biochim Biophys Acta 1125, 171-179
44. Oelkers, P., Behari, A., Cromley, D., Billheimer, J. T., and Sturley, S. L. (1998) J Biol Chem 273, 26765-26771
45. Kalscheuer, R., and Steinbuchel, A. (2003) J Biol Chem 278, 8075-8082
46. Tavani, D. M., Nes, W. R., and Billheimer, J. T. (1982) J Lipid Res 23, 774-781
47. Yang, H., Cromley, D., Wang, H., Billheimer, J. T., and Sturley, S. L. (1997) J. Biol. Chem. 272, 3980-3985
48. Lardizabel, K. D., Hawkins, D., and Thompson, G. (2000), US
49. Cheng, J. B., and Russell, D. W. (2004) J Biol Chem
50. Gunay-Aygun, M., Cassidy, S. B., and Nicholls, R. D. (1997) Behav Genet 27, 307-324
51. Vincent, M. C., Biancalana, V., Ginisty, D., Mandel, J. L., and Calvas, P. (2001) Eur J Hum Genet 9, 355-363
52. Lentes, K. U., Tu, N., Chen, H., Winnikes, U., Reinert, I., Marmann, G., and Pirke, K. M. (1999) J Recept Signal Transduct Res 19, 229-244
53. Surwit, R. S., Wang, S., Petro, A. E., Sanchis, D., Raimbault, S., Ricquier, D., and Collins, S. (1998) Proc Natl Acad Sci USA 95, 4061-4065
54. Katsanis, N., Lewis, R. A., Stockton, D. W., Mai, P. M., Baird, L., Beales, P. L., Leppert, M., and Lupski, J. R. (1999) Am J Hum Genet 65, 1672-1679
55. Saar, K., Geller, F., Ruschendorf, F., Reis, A., Friedel, S., Schauble, N., Nurnberg, P., Siegfried, W., Goldschmidt, H. P., Schafer, H., Ziegler, A., Remschmidt, H., Hinney, A., and Hebebrand, J. (2003) Pediatrics 111, 321-327
56. Li, W. D., Li, D., Wang, S., Zhang, S., Zhao, H., and Price, R. A. (2003) Diabetes 52, 1557-1561
57. Jiang, Y., Wilk, J. B., Borecki, I., Williamson, S., DeStefano, A. L., Xu, G., Liu, J., Ellison, R. C., Province, M., and Myers, R. H. (2004) Am J Hum Genet 75

1. A method for treating a subject afflicted with a disorder selected from the group consisting of a sebaceous gland disorder, obesity and atherosclerosis, comprising administering to the subject a therapeutically effective amount of an agent which inhibits acyl-CoA wax alcohol acyltransferase 1 (AWAT1) and/or acyl-CoA wax alcohol acyltransferase 2 (AWAT2), thereby treating the subject.

2. The method of claim 1, wherein the sebaceous gland disorder is selected from the group consisting of acne, rosacea, perioral dermatitis, sebaceous cysts, seborrhea and alopecia.

3. The method of claim 2, wherein the agent is administered topically.

4. The method of claim 1, wherein the agent selectively inhibits AWAT1.

5. The method of claim 1, wherein the agent selectively inhibits AWAT2.

6. The method of claim 1, wherein the agent is selected from the group consisting of magnesium, 2-bromooctanoate, an amidepsine, gemfibrozil, tetradecylthioacetic acid, isochromophilones, niacin, a tanshinone, cochiloquinones A and A1, fenofibrate, simvastatin, an n-3 polyunsaturated fatty acid, a phospholipid with a polar head group modified with monomethylethanolamine and/or dimethlyethanolamine, epidermal growth factor, a quinolone alkaloid, abscisic acid, an alkylaryl polyether alcohol, 2-bromopalmitate, 2-bromopalmitoyl-CoA, a lysophospholipid, glucagon, adrenaline, a chalcone, a roselipin, a prenylflavonoid, a polyacetylene, a benzoxiperone, and N-(7,10-dimetyl-11-oxo-10,11-dihydro-dibenzo[b,f][1,4]oxazepin-2-yl)-4-hydroxy-benzamide.

7. The method of claim 1, wherein the subject is a human.

8. A method for treating a subject afflicted with acne comprising topically administering to an afflicted area on the subject's skin a therapeutically effective amount of an agent which inhibits AWAT1 and/or AWAT2, thereby treating the subject.

9. The method of claim 8, wherein the agent selectively inhibits AWAT1.

10. The method of claim 8, wherein the agent selectively inhibits AWAT2.

11. The method of claim 8, wherein the agent is selected from the group consisting of magnesium, 2-bromooctanoate, an amidepsine, gemfibrozil, tetradecylthioacetic acid, isochromophilones, niacin, a tanshinone, cochiloquinones A and A1, fenofibrate, simvastatin, an n-3 polyunsaturated fatty acid, a phospholipid with a polar head group modified with monomethylethanolamine and/or dimethlyethanolamine, epidermal growth factor, a quinolone alkaloid, abscisic acid, an alkylaryl polyether alcohol, 2-bromopalmitate, 2-bromopalmitoyl-CoA, a lysophospholipid, glucagon, adrenaline, a chalcone, a roselipin, a prenylflavonoid, a polyacetylene, a benzoxiperone, and N-(7,10-dimetyl-11-oxo-10,11-dihydro-dibenzo[b,f][1,4]oxazepin-2-yl)-4-hydroxy-benzamide.

12. The method of claim 8, wherein the subject is a human.

13. A method for inhibiting the activity of AWAT1 and/or AWAT2 in a sebaceous gland comprising contacting the gland with a therapeutically effective amount of an agent which inhibits AWAT1 and/or AWAT2, thereby inhibiting the activity of AWAT1 and/or AWAT2 in the sebaceous gland.

14. The method of claim 13, wherein the agent selectively inhibits AWAT1.

15. The method of claim 13, wherein the agent selectively inhibits AWAT2.

16. The method of claim 13, wherein the agent is selected from the group consisting of magnesium, 2-bromooctanoate, an amidepsine, gemfibrozil, tetradecylthioacetic acid, isochromophilones, niacin, a tanshinone, cochiloquinones A and A1, fenofibrate, simvastatin, an n-3 polyunsaturated fatty acid, a phospholipid with a polar head group modified with monomethylethanolamine and/or dimethlyethanolamine, epidermal growth factor, a quinolone alkaloid, abscisic acid, an alkylaryl polyether alcohol, 2-bromopalmitate, 2-bromopalmitoyl-CoA, a lysophospholipid, glucagon, adrenaline, a chalcone, a roselipin, a prenylflavonoid, a polyacetylene, a benzoxiperone, and N-(7,10-dimetyl-11-oxo-10,11-dihydro-dibenzo[b,f][1,4]oxazepin-2-yl)-4-hydroxy-benzamide.

17. The method of claim 13, wherein the sebaceous gland is a human sebaceous gland.

18-44. (canceled)
